(19) United States
(12) Patent Application Publication
(10) Pub. No.: US 2002/0198231 A1
(43) Pub. Date: Dec. 26, 2002

(54) Compositions and methods for the treatment of Parkinson's disease

(76) Inventor: Jodi Nelson, Denver, CO (US)

Correspondence Address: GREENLEE WINNER AND SULLIVAN P C, 5370 MANHATTAN CIRCLE, SUITE 201, BOULDER, CO, 80303, US

(21) Appl. No.: 10/192,414

(22) Filed: Jul. 9, 2002

Related U.S. Application Data
(63) Continuation-in-part of application No. 09/615,639, filed on Jul. 13, 2000, now Pat. No. 6,417,177.
(60) Provisional application No. 60/143,767, filed on Jul. 13, 1999.
(60) Provisional application No. 60/175,051, filed on Jan. 7, 2000.
(60) Provisional application No. 60/202,140, filed on May 5, 2000.

(51) Int. Cl. A61K 31/4706

(52) U.S. Cl. 514/313

(57) ABSTRACT

This invention provides compositions and methods for increasing cellular respiration of melanized catecholamine neurons, and methods for alleviating symptoms or stopping appearance and/or progression of symptoms of Parkinson's disease and related conditions, characterized by nigrostriatal degeneration. An effective amount of a neuromelanin-binding composition having a quinoline ring in a suitable pharmaceutical carrier is administered to patient in need of such treatment. Preferably the composition comprises (−)-chloroquine. Selected adjuvants are also provided as part of the compositions of this invention.

This application is a continuation-in-part of U.S. patent application Ser. No. 09,615,639 filed Jul. 13, 2000, which takes priority from U.S. patent application Ser. No. 60/143,767 filed Jul. 13, 1999, U.S. patent application Ser. No. 60/175,051 filed Jan. 7, 2000, and U.S. patent application Ser. No. 60/202,140 filed May 5, 2000. All of the foregoing applications are incorporated herein by reference to the extent not inconsistent herewith.

### BACKGROUND

Idiopathic Parkinson's Disease (IPD) is a progressive neurodegenerative disorder. The onset of IPD symptoms begin to manifest when a threshold reduction of 60%-70% nigral neurons accompanied by an 80%-90% attenuation in striatal dopamine efflux, has been reached (Koller, W. C., “When does Parkinson's disease begin?”, (1992) Neurology 42(S4):27-31). Symptoms include tremor, postural imbalance, rigidity, bradykinesia and akinesia (Diagnostic Clinical Neuropsychology, Bigler, E. and Clement, P., Eds., 3^{rd} Ed. 1997). These symptoms intensify as the disease progresses. In severe stages of IPD, following the onset of akinesia, even the simplest movements require a monumental degree of concentration and mental effort, often to the point of anguish (Textbook of Medical Physiology, Guyton, A. C. and Hall, J. E., Eds., 9^{th} Ed., W. B. Saunders Company, Philadelphia, Pa., 1996). IPD is also characterized by a number of autonomic (Vainshtok, A. B., “Treatment of Parkinsonism with delagil,” (1972) Klin. Med (Mosk) 50(9):51-56) and non-motor symptoms including depression (Cummings, J. L., “Depression and Parkinson's Disease: A Review,” (1992) Am. J. Psychiatry 149(4):443-454) and frontal lobe dysfunction (Gotham, A. M. et al., “Levodopa treatment may benefit or impair ‘frontal’ function in Parkinson's disease,” (1986) Lancet 25;2(8513):970-971).

In the United States, it is estimated that 5-24 in every 100,000 people suffer from IPD, with the majority of low-income cases going undiagnosed (Chrischilles, E. A. et al., “The health burdens of Parkinson's disease,” (1998) Movement Disorders 13(3):406-413). In 1995, the World Health Organization (WHO) conducted a global epidemiological evaluation of the incidence of IPD, showing a worldwide incidence of 5.32 per 100,000 people with an astounding incidence rate of 49.33 per 100,000 people over the age of 65 (M. Privett, WHO). Although more recent epidemiological figures are unavailable, in 1996 with the world population being approximately 5.7 billion, an estimated 2.8 million people had a confirmed diagnosis of IPD.

Current pharmacological treatments for IPD and other Parkinsonian-like motor disorders include anticholinergic agents, catechol-o-methyltransferase inhibitors and dopaminergic agents (Physicians' Desk Reference, 2000, 54^{th} Ed., Medical Economics Company, Inc., Montvale, N.J.). Since the late sixties, dopamine precursor L-DOPA has been employed for the symptomatic relief of IPD motor dysfunction (Mena, M. A. et al., “Pharmacokinctics of L-DOPA in patients with Parkinson's disease,” (1986) Advances in Neurology 45:481-486). However, following long term use of L-DOPA (generally 5-8 years), diminished therapeutic efficacy is observed in approximately 50% of IPD patients (Roos, R. A. et al., “Response fluctuations in Parkinson's disease,” (1990) Neurology 40(9):1344-1346). A wearing off of L-DOPA efficacy precedes the development of serious motor side effects such as on/off motor oscillations and dyskinesias (Carlsson, Arvid, “Development of new pharmacological approaches in Parkinson's disease,” (1986) Advances in Neurology 45:513-518). Further, when medications are increased to compensate for the development of these new motor dysfunctions, more serious side effects are generally observed, including psychiatric complications, while producing only minimal therapeutic benefit (Stoof, J. C. et al., “Leads for the development of neuroprotective treatment in Parkinson's disease and imaging methods for estimating treatment efficacy,” (1999) Eur. J. Pharmacol. 375(1-3):75-86).

Deprenyl, a monoamine oxidase (MAO) B inhibitor, was the first drug suggested to provide causal treatment of Parkinson's Disease by alleviating symptoms and attenuating the progression of the illness (Mytilineou, C. et al., “L-(−)-desmethylselegiline, a metabolite of selegiline [L-(−)-deprenyl], protects mesencephalic dopamine neurons from excitotoxicity in vitro,” (1997) J. Neurochemistry 68(1):434-436). However, there remains much controversy regarding the therapeutic efficacy of Deprenyl. While some physicians prefer to prescribe Deprenyl when patients first present with symptoms of Parkinson's Disease (Goldstein, M. and Lieberman, A., “The role of the regulatory enzymes of catecholamine synthesis in Parkinson's disease,” (1992) Neurology 42(S4):8-12), other physicians dispute claims of neural protection (Olanow, C. W. and Calne, D., “Does selegiline monotherapy in Parkinson's disease act by symptomatic or protective mechanisms?” (1992) Neurology 42(S4): 13-26).

Poewe and Wenning (Poewe, W. H. and Wenning, G. K., “The natural history of Parkinson's disease,” (1998) Annals of Neurology 44(S1):S1-S9) reviewed several longitudinal studies that evaluated Parkinson's disease medications. The Parkinson's Research Group of the United Kingdom found that when Deprenyl was co-administered with L-DOPA, a 60% increase in patient mortality was observed, compared to the group being treated with L-DOPA only. Deprenyl is now rarely prescribed by European physicians for the treatment of IPD. In contrast, American researchers determined that Deprenyl is capable of delaying the need to commence L-DOPA treatment for a period of up to nine months. However, no neural protection was found in the two-year patient follow-up examinations (Poewe and Wenning, 1998, supra). In Parkinson's disease the average lifespan is 9.4 years following an initial diagnosis. Further, the onset of gait disorders is closely associated with mortality rate. To be superior to current causal treatments (i.e.—Deprenyl), a pharmacological treatment must: a) prolong the need to commence L-DOPA for more than nine months; b) retain efficacy beyond a two-year period; and, c) prevent, delay, or otherwise alleviate gait disorders.

Up to 20% of the people initially diagnosed with IPD actually suffer from atypical IPD (APD), striatonigral degeneration (SND), or multiple symptom atrophy (MSA) (Antonini, A. et al., “Differential diagnosis of Parkinsonism with [¹⁸F]Fluorodeoxyglucose and PET,” (1998) Movement Disorders 13(2):268-274). Little or no response to conventional Parkinson's disease drug therapy is usually the differentiating factor between a diagnosis of APD, SND and MSA as opposed to IPD (Dethy, S. et al., “Asymmetry of basal ganglia glucose metabolism and L-dopa responsiveness in Parkinsonism,” (1998) Movement Disorders 13(2):275-280). Often, little can be done for people suffering these atypical afflictions. Therefore, it would be of great benefit if a pharmacological means were identified that could alleviate symptoms of atypical Parkinson's disease, as well as IPD.

The exact cause or causes of IPD are still unknown. Nonetheless, scientists have discovered a multitude of pathological abnormalities in the Parkinsonian brain. These findings include but are not limited to: a) toxic metabolite formation during neuromelanin (NM) synthesis (Graham, D. G., “Autoxidation versus covalent binding of quinones as the mechanism of toxicity of dopamine, 6-hydroxydopamine, and related compounds toward C1300 neuroblastoma cells in vitro” (1978) Molecular Pharmacology 14:644-653); b) heightened affinity of endogenous and exogenous toxins for NM (Tipton, K. F. and Singer, T. P., “Advances in our understanding of the mechanisms of the neurotoxicity of MPTP and related compounds,” (1993) J. Neurochem. 61(4):1191-1206); c) mitochondrial impairment (Mizuno, Y. et al., “Mitochondrial dysfunction in Parkinson's disease,” (1998) Annals of Neurology 44(S1):S99-S109); d) increased oxidative stress potentiated by reduced levels of antioxidants (Merad-Boudia, M. et al., “Mitochondrial impairment as an early event in the process of apoptosis induced by glutathione depletion in neuronal cells: relevance to Parkinson's disease,” (1998) Biochem. Pharmacology 56:645-655); e) protein oxidation and lipid peroxidation (Jenner, P. et al., “Understanding cell death in Parkinson's disease,” (1998) Annals of Neurology 44(1):S72-S84); f) augmented iron content and abnormal Fe(II)/Fe(III) ratios (Riederer, P. et al., “Transition metals, ferritin, glutathione, and ascorbic acid in Parkinsonian brains,” (1989) J. Neurochemistry 52(2):515-520); and g) the accumulation of extracellular protein peptide fragments (Loo, D. T. et al., “Apoptosis is induced by β-amyloid in cultured central nervous system neurons,” (1993) Proc. Natl. Acad. Sci. USA 90:7951-7955). An example of these extracellular amyloid peptide fragments is the non-Aβ components in Lewy bodies (Culvenor, J. G. et al., “Non-Aβ component of Alzheimer's disease amyloid (NAC) revisited,” (1999) Am. J. Pathology 155:1173-1181) that trigger an apoptotic cascade. Taken individually, these pathological findings would not pose a tremendous cellular threat. Collectively and occurring simultaneously, they serve to progressively annihilate the melanized catecholamine neurons residing in the mesencephalon, ultimately producing the classic symptoms of Parkinson's Disease.

### Chloroquine Compounds

Chloroquine [7-chloro-4-(4-diethylamino-1-methylbutylamino)quinoline] (The Merck Index, p. 2220, 1996) is a synthetically manufactured anti-malarial containing the quinoline nucleus. Chloroquine was developed over fifty years ago. It continues to be the most widely employed drug for the treatment of the asexual erythrocytic form of P. falciparum (Deepalakshmi, P. D. et al., “Effect of chloroquine on rat liver mitochondria,” (1994) Indian J. Exp. Biology 32(11):797-799). Unfortunately, due to widespread use of chloroquine, treatment resistant strains of malaria, first reported in 1961, continue to emerge globally, making the use of chloroquine obsolete in many regions (Bitonti, A. J. et al., “Reversal of chloroquine resistance in malaria parasite plasmodium falciparum by desipramine,” (1988) Science 241:1301-1303). A number of chloroquine derivatives have been identified for antimalarial and other uses. See U.S. Pat. Nos. 5,948,791, 5,834,505, 5,736,557, 5,639,737, 5,624,938, 5,596,002 and 4,421,920.

Due to the widespread use of chloroquine, a number of beneficial therapeutic properties and medicinal applications, outside of conventional malarial treatment, have been identified. Most of the following studies employed chloroquine phosphate.

Chloroquine phosphate is a potent inhibitor of acid chondromucoprotease present in cartilage and of cathepsin B1, a protease especially important in the initiation of proteolysis (De Duve, C. et al., “Lysosomotropic agents,” (1974) Biochem. Pharm. 23:2495-2531). These properties have rendered chloroquine phosphate useful in the treatment of rheumatoid arthritis (Clinical Toxicology, supra, Section III, pp. 355-362). Further, chloroquine phosphate is known to reduce hypertension (Physician's Desk Reference, pp. 2301-2302, 1996). Chloroquine phosphate and other 4-aminoquinoline compounds have been prescribed for the treatment of cardiac arrhythmia (Clinical Toxicology, supra, Section III, pp. 355-362). A version of antimalarial drug called Cardioquin® is produced by the Purdue Fredrick Company for the maintenance of sinus rhythm after conversions from atrial fibrillation (Physician's Desk Reference, pp. 2521-2522, 2000). Chloroquine phosphate also targets malignant metastatic melanomas that generally bear an accumulation of melanin. The utilization of chloroquine phosphate for the treatment of these tumors is limited by the lack of accumulation in the amelanotic forms that readily manifest (Lindquist, N. G., “Accumulation of drugs on melanin,” (1973) Acta Radiol. Diag. (Stockholm) 325:1-92). A more recent application of chloroquine phosphate is to suppress the human immunodeficiency virus type 1 (HIV-1) replication in vivo within T-cells and monocytes (Sperber, K. et al., “Hydroxychloroquine treatment of patients with human immunodeficiency virus type 1,” (1995) Clinical Therapeutics 17(4):622-636; Ornstein, M. H. and Sperber, K., “The anti-inflammatory and antiviral effects of hydroxychloroquine in two patients with acquired immunodeficiency syndrome and active inflammatory arthritis,” (1996) Arthritis Rheum. 39(1):157-161).

The above medicinal applications of chloroquine phosphate are generally known to those practicing medicine in the USA. The following applications of chloroquine phosphate have been discovered and successfully employed by doctors throughout the world. Chloroquine phosphate has been used to treat renal disorders such as glomerulonephritis and amyloidosis, with observed improvement in renal function and attainment of various lengths of remission (Makarenko, I. E. and Levitsky, E. P., “Resoquin in the clinic of internal illnesses, and the possible side effects of its use,” (1950). A randomized trial of the prolonged use of chloroquine phosphate to treat advanced pulmonary sarcoidosis, suggests that patients responded better to chloroquine phosphate and withstood medication side effects better than with conventional corticosteroids (Baltzan, M. et al., “Randomized trial of prolonged chloroquine therapy in advanced pulmonary sarcoidosis,” (1999) Am. J. Respir. Crit. Care Med. 160:192-197). Conditions of acute hypertension can benefit from the administration of chloroquine phosphate, which acts as a vasodilator without depressing cardiac contractibility (Abiose, A. K. et al., “Chloroquine-induced venodilation in human hand veins,” (1997) Clin. Pharm. & Therapeutics 61(6):677-683). Chloroquine phosphate inhibits lysosomal proteolysis in vitro, and has been suggested to be a useful agent in counteracting protein wasting observed in several catabolic diseases (De Feo, P. et al., “Chloroquine reduces whole body proteolysis in humans,” (1994) Am. J. Physiology 267:E183-E186). Chloroquine phosphate inhibits the biological availability of iron and has been suggested as advantageous for the treatment of iron-loading disorders (Legssyer, R. et al., “Effect of chronic chloroquine administration on iron loading in the liver and reticuloendothelial system and on oxidative responses by the alveolar macrophages,” (1999) Biochem. Pharmacology 57(8):907-911). Several case histories have been published regarding the efficacy in administering chloroquine phosphate to infants suffering from desquamative interstitial pneumonitis who presented with failure to thrive, tachypnea and hypoxia (Springer, C. et al., “Chloroquine treatment in desquamative interstitial pneumonia,” (1987) Archives of Disease in Childhood 62:76-77), and were non-respondent to steroids (Leahy, F. et al., “Desquamative interstitial pneumonia responsive to chloroquine,” (1985) Clinical Pediatrics 24(4):230-232).

Further recognized, but non-FDA approved, uses of chloroquine phosphate include treatments for: cholera, idiopathic pulmonary hemosiderosis, lupus erythematosus, lymphoid interstitial pneumonitis, onchocerca volvulus, porphyria cutanea tarda, sarcoidosis, and ulcerative colitis (MICROMEXEX®, 2000, available: http//phamtom.uchsc.edu/mdxcgi/di). U.S. Pat. No. 5,430,039 suggests the use of chloroquine to inhibit neuronal cell death resulting from a calcium-related disorder of the central or peripheral nervous system, erroneously characterizing Parkinson's Disease as such a disorder. Vainshtok, A. B., “Treatment of Parkinsonism with delagil,” (1972) Klin. Med (Mosk) 50(9):51-56, reports administration of Delagil, a chloroquine compound or analog (exact compound unknown) to a group of medication-free patients (dosage unknown) suffering symptoms related to Parkinson's disease with moderate to dramatic response.

### Enantiomers of Chloroquine

Chloroquine and hydroxychloroquine are racemic mixtures of (−)- and (+)-enantiomers. The (−)-enantiomers are also known as (R)-enantiomers (physical rotation) and 1-enantiomers (optical rotation). The (+)-enantiomers are also known as (S)-enantiomers (physical rotation) and d-enantiomers (optical rotation). The (+)- enantiomer metabolizes peripherally about eight times more rapidly than the (−)-enantiomer, producing toxic metabolites including de-ethyl chloroquine (Augustijins, P. and Verbeke, N. [1993] “Stereoselective pharmacokinetic properties of chloroquine and de-ethyl chloroquine in humans,” Clinical Pharmacokinetics 24(3):259-69; Augustijins, P. et al. [1999], “Stereoselective de-ethylation of chloroquine in rat liver microsomes,” Eur. J. Drug Metabolism & Pharmacokinetics 24(1):105-8; DuCharme, J. and Farinotti R. [1996], “Clinical pharmacokinetics and metabolism of chloroquine,” Clinical Pharmacokinetics 31(4):257-74). Administering (+)-chloroquine may cause cardiac side effects due to toxic metabolite formation. The (−)-enantiomer has a longer half-life and lower clearance than the (+)-enantiomer (Ducharme, J. et al. [1995), “Enantio-selective disposition of hydroxychloroquine after a single oral dose of the racemate to healthy subjects,” British J. Clinical Pharmacology 40(2):127-33). The enantiomers of chloroquine and hydroxychloroquine may be prepared by procedures known to the art.

All publications referred to herein are incorporated by reference to the extent not inconsistent herewith.

### SUMMARY OF THE INVENTION

This invention provides compositions and methods for increasing cellular respiration of melanized catecholamine neurons such as dopamine neurons in the substantia nigra and basal ganglion, epinephrine and norepinephrine neurons, of protecting such neurons against oxidative degradation, and for treatment of Parkinson's Disease and related conditions, including both alleviation of symptoms and preventing onset or progression of symptoms. The compositions of this invention may be administered long-term. The compositions of this invention are also useful for preventing on-off syndrome, a condition in which L-Dopa and other dopamine agonists temporarily or permanently lose their ability to ameliorate the symptoms of Parkinson's disease after an initial period of effectiveness.

The term “Parkinson's Disease and related conditions” as used herein includes idiopathic Parkinson's Disease (IPD), atypical Parkinson's Disease (APD), non-L-dopa-responsive atypical Parkinson's Disease, Parkinson's Plus syndromes (which include supernuclear palsy and other non L-dopa responsive Parkinson's-type diseases), striatonigral degeneration (SND), multiple symptom atrophy (MSA), and vascular Parkinson's Disease.

This invention provides compositions useful for increasing cellular respiration of melanized catecholamine neurons comprising an active ingredient as described below, racemic mixtures, and enantiomers thereof, covalently linked, mixed, or complexed with an adjuvant, acceptable pharmaceutical salts thereof, and mixtures of the foregoing, said active ingredient and adjuvant being present in amounts effective to increase melanized catecholamine neurons.

The active ingredient is preferably chloroquine or a related compound (referred to herein as “CQ.” The term “CQ” includes chloroquine (7-chloro-4-(4-diethylamino-1-methylbutylamino)quinoline), chloroquine phosphate (7-chloro-4-(4-diethylamino-1-methylbutylamino) quinoline phosphate, and hydroxychloroquine (7-chloro-4-(4-diethylamino-1-methylbutylamino) quinoline), racemic mixtures, enantiomers, suitable pharmaceutical salts thereof, and mixtures thereof. Similarly, the terms (−)-chloroquine and (+)-chloroquine include (−)- and (+)-chloroquine phosphate and (−)- and (+)-hydroxychloroquine respectively.

The active ingredient may also be selected from compounds as above wherein hydrogen or fluorine is substituted for the chlorine atom on the molecule, e.g. 7-fluoro-4-(4-diethylamino-1-methylbutylamino)quinoline, 4-(4-diethylamino-1-methylbutylamino)quinoline, 7-fluoro-4-(4-diethylamino-1-methylbutylamino) quinoline phosphate, 4-(4-diethylamino-1-methylbutylamino) quinoline phosphate 7-fluoro-4-(4-diethylamino-1-methylbutylamino quinoline, and 4-(4-diethylamino-1-methylbutylamino quinoline, racemic mixtures, enantiomers, suitable pharmaceutical salts thereof, and mixtures thereof. Similarly, the terms (−)-enantiomer and (+)-enantiomer include (−)- and (+)-enantiomer phosphate and (−)- and (+)-hydroxy analogs of the foregoing respectively.

Compositions useful for increasing cellular respiration of melanized catecholamine neurons, and/or alleviating, preventing or halting progress of Parkinson's symptoms also may comprise, as active ingredients, neuromelanin-binding chloroquine and fluorine analogs and derivatives containing a quinoline nucleus, preferably selected from the group consisting of:

7-chloro-4-(4-diethylamino-1-methylbutylamino)quinoline (chloroquine);

7-fluoro-4-(4-diethylamino-1-methylbutylamino)quinoline;

4-(4-diethylamino-1-methylbutylamino)quinoline;

7-hydroxy-4-(4-diethylamino-1-methylbutylamino)quinoline;

chloroquine phosphate;

7-chloro-4-(4-diethylamino-1-butylamino)quinoline (desmethylchloroquine);

7-fluoro-4-(4-diethylamino-1-butylamino)quinoline);

4-(4-diethylamino-1-butylamino)quinoline;

7-hydroxy-4-(4-diethylamino-1-butylamino)quinoline;

7-chloro-4-(1-carboxy-4-diethylamino-1-butylamino)quinoline;

7-fluoro-4-(1-carboxy-4-diethylamino-1-butylamino)quinoline;

4-(1-carboxy-4-diethylamino-1-butylamino) quinoline;

7-hydroxy-4-(1-carboxy-4-diethylamino-1-butylamino)quinoline;

7-chloro-4-(1-carboxy-4-diethylamino-1-methylbutylamino)quinoline;

7-fluoro-4-(1-carboxy-4-diethylamino-1-methylbutylamino)quinoline;

4-(1-carboxy-4-diethylamino-1-methyl butylamino)quinoline;

7-hydroxy-4-(1-carboxy-4-diethylamino-1-methylbutylamino)quinoline;

7-chloro-4-(4-ethyl-(2-hydroxyethyl)-amino-1-methylbutylamino)quinoline (hydroxychloroquine);

7-fluoro-4-(4-ethyl-(2-hydroxyethyl)-amino-1-methylbutylamino)quinoline;

4-(4-ethyl-(2-hydroxyethyl)-amino-1-methylbutylamino)quinoline 7-hydroxy-4-(4-ethyl-(2-hydroxyethyl)-amino-1-methylbutylamino)quinoline;

hydroxychloroquine phosphate;

7-chloro-4-(4-ethyl-(2-hydroxyethyl)-amino-1-butylamino)quinoline (desmethylhydroxychloroquine);

7-fluoro-4-(4-ethyl-(2-hydroxyethyl)-amino-1-butylamino)quinoline;

4-(4-ethyl-(2-hydroxyethyl)-amino-1-butylamino)quinoline;

7-hydroxy-4-(4-ethyl-(2-hydroxyethyl)-amino-1-butylamino)quinoline;

7-chloro-4-(1-carboxy-4-ethyl-(2-hydroxyethyl)-amino-1-butylamino)quinoline;

7-fluoro-4-(1-carboxy-4-ethyl-(2-hydroxyethyl)-amino-1-butylamino)quinoline;

4-(1-carboxy-4-ethyl-(2-hydroxyethyl)-amino-1-butylamino)quinoline;

7-hydroxy-4-(1-carboxy-4-ethyl-(2-hydroxyethyl)-amino-1-butylamino)quinoline;

7-chloro-4-(1-carboxy-4-ethyl-(2-hydroxyethyl)-amino-1-methylbutylamino)quinoline;

7-fluoro-4-(1-carboxy-4-ethyl-(2-hydroxyethyl)-amino-1-methylbutylamino)quinoline;

4-(1-carboxy-4-ethyl-(2-hydroxyethyl)-amino-1-methylbutylamino)quinoline;

7-hydroxy-4-(1-carboxy-4-ethyl-(2-hydroxyethyl)-amino-1-methylbutylamino)quinoline;

8-[(4-aminopentyl)amino)-6-methoxydihydrochloride quinoline;

1-acetyl-1,2,3,4-tetrahydroquinoline; 8-[4-aminopentyl)amino]-6-methoxyquinoline dihydrochloride;

1-butyryl-1,2,3,4-tetrahydroquinoline; 7-chloro-2-(o-chlorostyryl)-4-[4-diethylamino-1-methylbutyl]aminoquiinoline phosphate;

3-chloro-4-(4-hydroxy-α,α′-bis(2-methyl-1-pyrrolidinyl)-2,5-xylidinoquinoline, 4-[(4-diethylamino)-1-methylbutyl)amino] -6-methoxyquinoline;

3-fluoro-4-(4-hydroxy-α,α′-bis(2-methyl-1-pyrrolidinyl)-2,5-xylidinoquinoline, 4-[(4-diethylamino)-1-methylbutyl)amino]-6-methoxyquinoline;

4-(4-hydroxy-α,α′-bis(2-methyl-1-pyrrolidinyl)-2,5-xylidinoquinoline, 4-[(4-diethylamino)-1-methylbutyl)amino]-6-methoxyquinoline;

3,4-dihydro-1-(2H)-quinolinecarboxyaldehyde;

1,1′-pentamethylenediquinoleinium diiodide; and 8-quinolinol sulfate, racemic mixtures, and enantiomers thereof, and suitable pharmaceutical salts thereof including phosphate salts of all the foregoing compounds, said compounds covalently linked or complexed or mixed with adjuvants and mixtures thereof, as well as suitable pharmaceutical salts thereof. Chloroquine and hydroxychloroquine are preferred; (−)-enantiomers thereof are more preferred, and said compounds covalently linked or complexed or mixed with adjuvants are most preferred. Neuromelanin-binding compounds such as chlorpromazine and other antipsychotics, which bind to dopamine receptors, are not included within the scope of PD-effective neuromelanin-binding compounds of this invention. Any chloroquine analog or derivative known to the art and capable of binding neuromelanin may be useful in the methods of this invention.

The neuromelanin-binding compound may be selected from the group consisting of compounds capable of crossing the blood-brain barrier in effective amounts. Such compounds include those which are more lipophilic, are capable of changing to effective chirality after crossing the blood-brain barrier, have side chain substituents which enhance compound transport via blood-brain barrier transporter mechanisms, or are complexed or covalently linked with antibodies or other targeting moieties, or administered in combination with other compounds facilitating their crossing the blood-brain barrier, as known to the art. The (−)-enantiomer of chloroquine (referred to herein as the active enantiomer) is preferred.

In a preferred embodiment, the compositions useful for increasing cellular respiration of melanized catecholamine neurons comprise an effective amount of a composition comprising (−)-CQ or (−)-CQ mixed, complexed, or covalently linked with an adjuvant; an amount of (+)-CQ less than that of said (−)-CQ or (−)-CQ mixed, complexed, or covalently linked with a targeting agent; and a suitable pharmaceutical carrier.

When CQ enantiomers are administered separately, there is significantly less CQ accumulation in the eyes, and thus less CQ-associated retinal degeneration.

Such compositions containing (−)-chloroquine may include anywhere from no (+)-CQ to about 49% (+)-CQ. An amount of (+)-CQ sufficient to bind to enzymes causing peripheral breakdown of CQ is preferred, leaving more of the (−)-CQ to cross the blood brain barrier where its therapeutic effect takes place. Preferably the compositions comprise between about 10% and about 20% (+)-CQ.

Adjuvants herein are preferably selected from the group consisting of peripheral membrane protective agents, such as retinal protective agents, peripheral metabolism inhibitors which inhibit peripheral metabolism of the active ingredient, enhancing agents such as histamine H₁ receptor antagonists, neural protective compounds other than the active ingredients as defined herein, dopamine and dopamine agonists, free radical deactivators, and antioxidants.

A targeting agent is a substance that when complexed with the active ingredient helps carry it across the blood brain barrier. Preferred targeting agents are lipophilic moieties known to the art which are attached to the active molecule at a position which does not interfere with the ability of the quinoline ring to bind to neuromelanin, and antibodies such as an antibody capable of binding to lactotransferrin receptors pathologically expressed on the vasculature in close anatomical proximity to the mesencephalon. Using such lactotransferrin antibodies covalently attached to the active ingredient, preferably covalently attached to chloroquine, chloroquine phosphate, or hydroxychloroquine, competitively inhibits the incorporation of iron into the neurons, and thereby attenuates the pathological incorporation of iron which has been characterized as being contributory to oxidation stress and subsequent neural degeneration in Parkinson's.

Retinal and peripheral membrane protective agents (also called “protectors”) are desirable when the active ingredient is administered long-term, e.g. for a year or more. Chloroquine and related compounds tend to bind to membranes and cause rigidity in the membranes, especially mitochondrial membranes. CQ is a calcium ion ATPase pump inhibitor. In the retina, CQ binds to pigment and produces retinal degeneration. Peripheral membrane protective agents also act to counteract peripheral sympathetic nerve damage occurring in Parkinson's disease, by means of their membrane-stabilizing and neural protective activities.

The compositions of this invention also act as effective agents to counteract loss of sympathetic neuron efferents and attenuated norepinephrine by inducing a supersensitivity to endogenously lower the response threshold to effectors governed by norepinephrine sympathetic neuron fibers.

Preferred retinal and peripheral membrane protectors are selected from the group consisting of calcium citrate, calcium gluconate, calcium lactate, and calcium phosphate, but not calcium carbonate. Preferably the retinal and peripheral membrane protector includes Vitamin D to facilitate gastrointestinal absorption of the calcium. Calcium ions have a high affinity to retinal melanin, accumulate in the eye pigment and competitively inhibit CQ binding to retinal melanin. Also, increasing calcium ion concentration can help restore flexibility to other membranes, especially mitochondrial membranes. Since calcium ions compete with CQ for binding membrane and melanin binding sites, it is preferred that the calcium ions be administered along with the active ingredient in a time-release formulation wherein the calcium ions are released about two to three hours prior to CQ release.

Peripheral metabolism inhibitors are compounds that inhibit breakdown of active ingredients into their metabolites (e.g., for chloroquine and its enantiomers, monodesethylchloroquine and desethylchloroquine and their enantiomers), and thereby increase the active ingredient availability for crossing the blood-brain barrier where it is active for the therapeutic purposes of this invention. CQ is generally more lipophilic than its metabolites, and thus more easily crosses the blood-brain barrier. Use of peripheral metabolism inhibitors can allow dosage reduction of the active ingredient by allowing greater active ingredient incorporation into the central nervous system, and therefore, better Parkinson's treatment efficacy. For example, CQ dosages can be reduced to as low as about 100 mg to 200 mg base equivalents daily.

Peripheral metabolism inhibitors may also serve as retinal protective agents since CQ metabolites more readily bind to eye pigment than CQ itself, and reducing the amount of available metabolites will reduce the amount of retinal degeneration.

The use of such peripheral metabolism inhibitors also helps lower incidence of cardiac and dermatological adverse events associated with CQ metabolites, thereby improving the safety and toxicology profiles of compositions described herein, especially when compared with standard antimalarial agents.

Preferred peripheral metabolism inhibitors are cytochrome P450 2D6 and/or 3A enzyme inhibitors. These can reduce the amount of CQ metabolites present. These inhibitors do not prevent absorption of dopamine, L-dopa or other dopamine agonists, but may interfere with bioavailability of other medications a patient may be taking, and if so, it is preferred that the compositions of this invention containing such P450 inhibitors be administered in the evening, or at another time when the medications they interfere with will not be administered.

Preferred cytochrome (CYP) 2D6 enzyme inhibitors are those selected from the group consisting of amiodarone, celecoxib, chlorpheniramine, cimetidine, clomipramine, fluoxetine, levomepromazine, metoclopramide, mibefradil, moclobemide, paroxetine, quinidine, ranitidine, ritonavir, sertraline, terbinafine, racemic mixtures and enantiomers, and suitable pharmaceutical salts of the foregoing.

Preferred daily dosage amounts of the foregoing cytochrome (CYP) 2D6 enzyme inhibitors are as follows:

Amiodarone: about 400 mg to about 800 mg. This is a preferred adjuvant because it acts as an inhibitor upon both CYP 2D6 and CYP 3A.

Celecoxib: about 200 mg to about 400 mg. This adjuvant is known as an antiarthritic agent, and is preferred for use when treating patients having concurrent arthritis.

Chloropheniramine: about 6 mg to about 10 mg. This adjuvant is a histamine H₁ receptor antagonist as discussed below.

Cimetidine: about 400 mg to about 600 mg. Cimetidine is a well-known anti-ulcer and anti-acid reflux agent and is preferably used when treating patients having concurrent gastrointestinal problems, or gastrointestinal problems caused by administration of CQ or other active ingredient. Cimetidine has been used in clinical studies of compositions of this invention with good results, and is a preferable adjuvant due to its absence of adverse cardiac and hypotensive effects.

Clomipramine: about 25 mg to about 100 mg. This is an antidepressant and is preferred when treating patients having concurrent clinical depression.

Fluoxetine: about 20 mg to about 60 mg. This is also an antidepressant and preferred when treating patients having clinical depression.

Levomepromazine: about 15 mg to about 35 mg.

Metoclopramide: about 25 mg to about 30 mg. Like cimetidine, this is an anti-ulcer and anti-acid reflux agent and is preferably used when treating patients having concurrent gastrointestinal problems, or gastrointestinal problems caused by administration of CQ or other active ingredient.

Mibefradil: about 25 mg to about 50 mg.

Moclobemide: about 200 mg to about 30 mg. Moclobemide is an antidepressant, preferably used for treating patients with concurrent clinical depression.

Paroxetine: about 20 mg to about 40 mg. Paroxetine is also an antidepressant, preferably used for treating patients with concurrent clinical depression.

Quinidine: about 200 mg to about 400 mg.

Ranitidine: about 200 mg to about 300 mg. Ranitidine is an anti-ulcer and anti-acid reflux agent and is preferably used when treating patients having concurrent gastrointestinal problems, or gastrointestinal problems caused by administration of CQ or other active ingredient.

Ritonavir: about 600 mg to about 1200 mg.

Sertraline: about 25 mg to about 50 mg. Sertraline is an antidepressant, preferably used for treating patients with concurrent clinical depression.

Terbinafine: about 200 mg to about 400 mg.

Preferred cytochrome P450 3A enzyme inhibitors are those selected from the group consisting of delaviridine, indinavir, nelfinavir, saquinavir, amiodarone, cimetidine, ciprofloxacin, clarithromycin, diethyl-dithiocarbamate, diltiazem, erythromycin, fluconazole, fluvoxamine, itraconazole, ketoconazole, mifepristone, nefazodone, norfloxacinem, norfluoxetine, racemic mixtures and enantiomers, and suitable pharmaceutical salts of the foregoing.

Preferred daily dosage amounts of the foregoing cytochrome (CYP) 3A enzyme inhibitors are as follows:

Amiodarone: about 400 mg to about 800 mg. This is a preferred adjuvant because it acts as an inhibitor upon both CYP 2D6 and CYP 3A.

Delaviridine: about 400 mg to about 1200 mg.

Indinavir: about 600 mg to about 1200 mg.

Nelfinavir: about 600 mg to about 1200 mg.

Saquinavir: about 1000 mg to about 2000 mg.

Amiodarone: about 400 mg to about 800 mg.

Cimetidine: about 400 mg to about 600 mg.

Ciprofloxacin: about 200 mg to about 200 mg.

Clarithromycin: about 200 mg to about 400 mg.

Diethyl-dithiocarbamate: about 10 mg to about 1000 mg. This compound (carbamic acid) is a metal ion-chelating agent currently being tested for its ability to slow progression of AIDS. As a chelating agent capable of binding iron ions, this is a highly preferred agent for use as a CYP 3A inhibitor.

Diliazem: about 5 mg to about 15 mg.

Erythromycin: about 500 mg to about 1000 mg.

Fluconazole: about 200 mg to about 400 mg.

Fluvoxamine: about 50 mg to about 100 mg. Fluvoxamine is an antidepressant, preferably used for treating patients with concurrent clinical depression.

Itraconazole: about 200 mg to about 400 mg.

Ketoconazole: about 200 mg to about 400 mg.

Mifepristone: about 25 mg to about 50 mg.

Nefazodone: about 50 mg to about 150 mg. Nefazodone is an antidepressant, preferably used for treating patients with concurrent clinical depression.

Mifepristone: about 2000 mg to about 3000 mg.

Norfloxacin: about 250 mg to about 500 mg.

Norfluoxetine: about 40 mg to about 100 mg. Norfluoxetine, like fluoxetine, is an antidepressant, preferably used for treating patients with concurrent clinical depression. Most preferably, a combination of fluoxetine and norfluoxetine is used in formulas for severely depressed Parkinson's patients.

Preferably, the peripheral metabolism inhibitors are administered along with the active ingredients of this invention in the form of a time-release preparation wherein the inhibitors are released about one and a half to about two hours after the retinal and peripheral protective agents, and about one hour before the active ingredient to maximize gastrointestinal absorption and enhance pharmacodynamic interactions.

Enhancing agents are agents, which act to increase levels of active ingredient in the brain or to increase dopamine levels in the brain. Preferred enhancing agents are histamine (H₁) receptor antagonists. These act to counteract increased histamine bioavailability resulting from active ingredient, especially CQ, inhibition of histamine methyltransferase (HMT) and diamine oxidase (DAO, the two primary degradative histamine pathways by chloroquine, and to minimize histamine-associated adverse events, which have been observed with antimalarial treatment formulas.

Although studies have shown that CQ is able to inhibit the action of histamine at certain receptors (i.e., in asthma studies, bronchial arterioles), one of the major problems seen in the treatment of malaria with CQ is pruritis. Pruritis is a histamine-invoked dermatological problem that occurs in about 35% of people being treated for malaria with CQ. It is easily treated with Hiantagonists like chlorpheniramine or Benadryl, so it is clear that CQ does not inhibit this receptor type peripherally. It is best to treat pruritis by administering an antihistamine before administering CQ, especially when treating patients who are very sensitive to CQ metabolites that, in addition to cardiac side effects, contribute more to the generation of pruritis than parent CQ molecules.

One embodiment of this invention utilizes first-generation histamine H₁ receptor antagonists as adjuvants. First-generation histamine H₁ receptor antagonists are those that are capable of crossing the blood-brain barrier. These agents can cause drowsiness. Such first-generation histamine H₁ receptor antagonists are preferably selected from the group consisting of carbinoxamine maleate, clemastine, diphenhydramine, dimenhydrinate, pyrilamine maleate, tripelennamine, chlorpheniramine maleate, brompheniramine maleate, hydroxyzine hydrochloride, hydroxyzine pamoate, cyclizine hydrochloride, cyclizine lactate, meclizine hydrochloride, promethazine hydrochloride, and racemic mixtures and enantiomers and suitable pharmaceutical salts of the therapeutic moieties of the foregoing. Other pharmaceutically effective salts of the foregoing compounds than those mentioned above are also useful.

Preferred daily dosages for the foregoing first-generation histamine H₁ receptor antagonists are as follows:

Carbinoxamine maleate: about 10 mg to about 8 mg.

Clemastine: about 3 mg to about 6 mg.

Diphenhydramine: about 50 mg to about 100 mg.

Dimenhydrinate: about 100 mg to about 200 mg.

Pyrilamine maleate: about 100 mg to about 200 mg.

Tripelennamine: about 100 mg-preferably in sustained release form.

Chlorpheniramine maleate: about 12 mg.-preferably in sustained release form. Other chlorpheniramine salts may also be used. Preferably, the chlorpheniramine is administered in the form of d-chlorpheniramine, as this form has higher efficacy than the 1-form or the racemic form, and use of a more effective form can save space in a capsule in which the composition is packaged.

Brompheniramine maleate: about 12 mg sustained release.

Hydroxyzine hydrochloride: about 50 mg to about 100 mg.

Hydroxyzine pamoate: about 50 mg to about 100 mg.

Cyclizine lactate: about 50 mg to about 100 mg.

Mecllizine hydrochloride: about 40 mg to about 60 mg.

Promethazine hydrochloride: about 50 mg to about 100 mg.

In another embodiment of this invention, a second-generation histamine (H₁) receptor antagonist is used as an adjuvant. Second-generation histamine (H₁) receptor antagonists are not capable of crossing the blood-brain barrier, and therefore do not cause drowsiness. Preferred second-generation histamine (H₁) receptor antagonists are those that do not cause adverse cardiac effects, e.g. torsaides des pointes and arrhythmias.

Preferred second-generation histamine (H₁) receptor antagonists for use as adjuvants herein are selected from the group consisting of acrivastine, cetirizine hydrochloride, astemizole, loratadine and terfenadine, racemic mixtures and enantiomers thereof, and acceptable pharmaceutical salts of the therapeutic moieties of the foregoing.

Preferred daily dosages for second-generation histamine (H₁) receptor antagonists are as follows:

Acrivastine: about 15 mg to about 25 mg.

Cetirizine hydrochloride: about 10 mg to about 20 mg.

Astemizole: about 10 mg.

Loratadine: about 5 mg to about 10 mg.

Terfenadine: about 60 mg.

Compositions of this invention comprising enhancing agents may be prepared in the form of time-release preparations. Preferably the enhancing agent is released concurrently with the active ingredient.

Compositions comprising enhancing agents are capable of affording neuroprotection and can prevent manifestation of Parkinson's disease motor symptoms if treatment is started early in the disease state, i.e., before about fifty percent of the dopamine neurons in the substantia nigra have been lost. Slowing of progression of Parkinson's disease is accomplished by the active ingredient being able to counteract the majority of pathological indices described as contributing to the neurodegeneration seen in Parkinson's Disease.

The foregoing compositions comprising enhancing agents are synergistic with other available Parkinson's disease medications and are capable of prolonging the utility and efficacy of other available Parkinson's disease medications by allowing patients to postpone taking L-Dopa and other available Parkinson's disease medications, allowing for dramatic dose reductions in concomitant Parkinson's disease medications when patients begin taking the compositions of this invention, and by slowing and/or arresting dopamine cell loss, making it no longer necessary to steadily increase dosages of currently-available Parkinson's disease medications.

Compositions of this invention may also comprise an effective amount of at least one adjuvant selected from the group consisting of antioxidants, other retinal protective agents, other neural protective compounds, dopamine or dopamine agonists, and free radical deactivators.

The antioxidant may be any antioxidant known to the art to prevent free radical formation and oxidative degradation of tissues and is preferably selected from the group consisting of probucol, pycnogenol, Vitamin C, Vitamin E, superoxide dismutase, preferably synthetic, BHT, BHA, and melatonin.

The retinal protective agent is a composition administered locally to prevent binding of retinal melanin with CQ, as is known to the art, e.g., alkanes and alcohols of C₁-C₄, ginko biloba and the calcium compounds and vitamin D adjuvants discussed above.

The neural protective compound is any compound known to the art and preferably is selected from the group consisting of selegiline hydrochloride and other monoamine oxidase inhibitors.

The dopamine agonist is any compound known to the art as an anti-Parkinson's treatment and preferably is selected from the group consisting of L-DOPA, pramipexole, ropinerole, bromocriptine, tolcapone, and carbidopa.

The free radical deactivator is any compound known to the art and preferably is selected from the group consisting of superoxide dismutase, selegiline, hydrochloride, and tolcapone.

The compositions of this invention are capable of augmenting dopamine availability, as seen in behavioral results generated in clinical studies thereof, by way of two primary mechanisms: First, CQ inhibits re-uptake of catecholamines including dopamine; and secondly, our analysis reveals that CQ is structurally compatible and pharmacokinetically related to two mixed monamine oxidase A and B inhibitors, namely hydralazine hydrochloride (CAS No. 304-20-1) and quinacrine dihydrochloride (CAS No. 69-05-6), and thus can inhibit degradation of catecholamines.

In one embodiment, a single adult dosage amount of said composition effective for increasing cellular respiration of melanized catecholamine neurons is provided. Active ingredients may be provided in dosages as high as will be tolerated, e.g. malarial dosages up to 500 mg per day, but preferably less than an antimalarial single adult dosage amounts are used, more preferably less than about 1 mM base equivalents, and most preferably less than about 0.5 mM base equivalents of CQ. As is known to the art, the term “base equivalents” refers to amount of active ingredient (e.g., in reference to chloroquine phosphate, refers to the chloroquine minus the phosphate and filler components). A single adult dosage amount with respect to use for alleviation, preventing or stopping progression of symptoms of Parkinson's disease or for other uses described herein will be an amount effective when administered daily to provide the stated therapeutic effect. Compositions of this invention comprising adjuvants that increase the bioavailability of the active ingredient may be administered in active-ingredient dosages as low as about 100 mg to about 200 mg daily.

This invention also provides kits comprising in close proximity, such as in a container or blister pack, effective dosage amounts and forms of the compositions of this invention for single doses, or doses per week, or other appropriate time period, preferably in combination with an adjuvant, such as a peripheral retinal or membrane protective agent, a peripheral metabolism inhibitor, an enhancing agent, an antioxidant, dopamine or dopamine agonist, free radical deactivation, or other adjuvant as discussed above suitable for co-administration with said composition, in effective dosage forms and amounts.

Suitable pharmaceutical carriers are known to the art and include carriers aiding in transport across the blood/brain barrier, such as nanoparticles onto which the compositions are absorbed, coated with a detergent, e.g., as described in Begley, D. J. (1996) “The blood-brain barrier: principles for targeting peptides and drugs to the central nervous system,” J. Pharm. Pharmacol. 48(2):136-46, incorporated herein by reference to the extent not inconsistent herewith.

This invention also provides methods for increasing cellular respiration of melanized catecholamine neurons, and methods for alleviating symptoms or stopping appearance and/or progression of symptoms of Parkinson's and related diseases, and methods for preventing symptoms of on-off syndrome associated with treatment with dopamine or a dopamine agonist, of a patient suffering symptoms of a disease selected from the group consisting of idiopathic and atypical Parkinson's disease, conditions characterized by nigrostriatal degeneration, multiple system atrophy, and vascular Parkinson's disease, as well as non-L-dopa-responsive atypical Parkinsonian disorders. sometimes called “Parkinson's plus syndrome.” Said methods comprise administering to said patient an effective amount of an above composition of this invention. The methods are suitable for any mammal having such melanized neurons or symptoms of Parkinson's disease. Methods for treating or preventing symptoms of Parkinson's Disease and related conditions as described above also comprise identifying patients having such symptoms or at risk of developing them.

Clinical studies have shown that compositions of this invention can effectively improve cognition, alleviate motor symptoms and attenuate the progression of Parkinson's disease and the foregoing related disorders when administered in dosages similar to dosages that are required to treat idiopathic Parkinson's disease.

Further provided herein are methods of making pharmaceutical compositions which are effective for increasing cellular respiration of melanized catecholamine neurons comprising: providing a composition of this invention as described above comprising an active ingredient and an adjuvant, providing a suitable pharmaceutical carrier; and mixing said composition and pharmaceutical carrier to form a composition effective to increase cellular respiration of melanized catecholamine neurons.

Instead of mixing (+)-CQ with (−)-CQ, the method of making the compositions of this invention comprising (−)-CQ may be practiced by starting with racemic chloroquine and removing an amount of (+)-CQ to leave a CQ composition effective to increase cellular respiration of melanized catecholamine neurons.

### DETAILED DESCRIPTION

The term “increasing cellular respiration” means measurably increasing oxygen consumption, increasing aerobic cellular respiration and reducing anaerobic cellular respiration, e.g., as measured by lactate in the cerebral spinal fluid.

The term “diminishing oxidative degradation of dopamine neurons in the substantia nigra and basal ganglion” means measurably diminishing such degradation as measured by assays known to the art, including measures of free iron ion availability, lipid peroxidation by-products such as malondialdehyde formation, and oxygenated radical formation.

The term “alleviating symptoms of Parkinson's disease or related conditions” means measurably reducing, inhibiting, attenuating and/or compensating for at least one symptom of Parkinson's disease or related condition, such as tremor, postural imbalance, rigidity, bradykinesia, akinesia, gait disorders, and on/off fluctuations. These symptoms may result from toxic metabolite formation during neuromelanin (NM) synthesis, heightened affinity of endogenous and exogenous toxins for NM, mitochondrial impairment, increased oxidative stress potentiated by reduced levels of antioxidants, protein oxidation and lipid peroxidation, augmented iron content and abnormal Fe(II)/Fe(III) ratios, and the accumulation of extracellular protein peptide fragments, which conditions may also be alleviated by the compositions of this invention.

The compositions of this invention containing (−)-CQ should have more (−)-CQ or (−)-CQ mixed, complexed, or covalently linked with an adjuvant than (+)-CQ because the toxic metabolites of (+)-CQ make it less suitable for long-term use, and the better melanin-binding properties of (−)-CQ, its longer half life and lower clearance make it more effective for long-term administration (e.g. at least about six weeks, more preferably, about two years, and most preferably, at least about ten years or more).

An effective amount of the compositions of this invention is an amount necessary to produce a measurable effect. For example, an effective amount of the compositions of this invention to increase cellular respiration measurably increases cellular respiration by assays known to the art as discussed above. In compositions containing (−)-CQ, the effect may be produced by the (−)-CQ, or partially by the (−)CQ and partially by (+) CQ. Similarly, an effective amount of a composition of this invention to alleviate or stop the progression of symptoms of Parkinson's Disease is an amount which does so based on art-known tests such as the Unified Parkinson's Disease Rating Scale and the Tinetti Gait and Balance Assessment Tool, comparing symptoms of treated patients with symptoms of the same patients prior to and/or after treatment, or with symptoms of untreated patients at the same stage of Parkinson's Disease.

Preventing symptoms of Parkinson's Disease includes identifying patients at risk for developing such symptoms. Identification of patients susceptible to onset of Parkinson's Disease may be done by genetic testing, prediction from family history or other means known to the art such as PET scans. When symptoms of Parkinson's do not develop, or do not develop to the expected (average) degree, they are considered to have been prevented by the methods and compositions of this invention.

Preventing on-off symptoms in patients being treated with L-Dopa or like medications means measurably stopping or decreasing such symptoms as compared with patients at similar stages of Parkinson's Disease being treated with such medications.

The compounds of this invention may be formulated neat or may be combined with one or more pharmaceutically acceptable carriers for administration, such as solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solution or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

The effective dosage of active ingredient employed may vary depending on the particular mixture employed, the mode of administration and the severity of the condition being treated. However, in general, satisfactory results are obtained when the active ingredients of the invention are administered at a daily adult dosage of from about 0.5 to about 1000 mg, optionally given in divided doses two to four times a day, or in sustained release form. For most large mammals the total daily dosage is from about 1 to 1000 mg, preferably from about 2 to 500 mg. Dosage forms suitable for internal use comprise from about 0.5 to 1000 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation. Preferably a single daily adult dose comprises less than about 1 mM, and more preferably less than about 0.5 mM base equivalents, more preferably less than about 1 mM, and more preferably less than about 0.5 mM base equivalents. Active-ingredient dosages of between about 100 mg and about 200 mg base equivalents daily may be used, especially in combination with adjuvants which increase bioavailability of the active ingredient as described above.

The compounds of this invention may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

The preferred pharmaceutical compositions from the standpoint of ease of preparation and administration are solid compositions, particularly tablets and hard-filled or liquid-filled capsules. Oral administration of the compositions is preferred. Time-release formulas as described above are desirable in many cases as taught herein. In some cases it may be desirable to administer the compounds to the patient's airways in the form of an aerosol.

The compounds of this invention may also be administered parenterally or intraperitoneally. Solutions or suspensions of these active compounds as a freebase or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparation contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

“Suitable pharmaceutical carriers” as referred to herein include distilled and pharmaceutical grade water, but do not include water or buffers unsuitable for administration to a human patient.

There are several mechanisms by which neuromelanin may contribute to symptoms of Parkinson's disease by contributing to formation of toxic products including superoxide and hydroxy radicals, which catalyze lipid peroxidation, and oxidation of NADH resulting in disruption of the neuron's respiration and reducing the amount of energy available to the neurons via aerobic respiration.

Neuromelanin can be considered a waste product of catecholamine degradation and gradually accumulates within the cytosol of catecholamine neurons throughout one's lifetime. Dopamine is autoxidized to cytotoxic and reactive oxygenated species such as 6-hydroxydopamine (6-OHDA) and semiquinone radicals. Low glutathione levels contribute to oxidative stress in Parkinson's disease, and allow available hydrogen peroxide to be further catalyzed by iron into highly toxic superoxide radicals and hydroxyl radical species as well as semiquinone radicals. Dopamine and L-DOPA interaction with superoxide radicals augments depletion of glutathione, leading to a downward spiral of detrimental reactions.

Monoamine oxidase forms toxic metabolites from a number of substances such as beta-carboline derivatives and tetrahydroisoquinoline that are present in excessive amounts in the cerebral spinal fluid of people with Parkinson's Disease. These toxic metabolites have high affinity to neuromelanin, and once bound may cause almost complete arrest of ATP production, resulting in impaired respiration, loss of energy available to the neurons and massive melanized cell loss which leads to symptoms of Parkinson's Disease. Inhibitors of monoamine oxidase B such as Deprenyl prevent formation of these toxic metabolites. Iron also tends to bind to neuromelanin, resulting in a cascade of pathogenic reactions leading to neuronal death. Increasing iron concentrations in basal ganglia are observed with normal aging, and in patients with Parkinson's Disease, iron is pathologically elevated with high ferric/ferrous ion ratios. The ferric ions contribute, with 6-OHDA, to the formation of harmful superoxide and hydroxyl radicals leading to lipid peroxidation and cell breakdown.

Iron chelators have been shown to reverse impaired mitochondrial respiration caused by 6-OHDA inhibition of NADH dehydrogenase. 6-OHDA catalyzes the release of iron from intracellular ferritin stores which in turn catalyzes lipid peroxidation. This toxic chain of events can be inhibited by superoxide dismutase. Both iron chelators and chloroquine phosphate have been found to limit the availability of free iron, so that it is not available to catalyze these toxic reactions.

The iron transporter protein, diferric transferrin, which delivers iron throughout the body also contributes to loss of energy available to the neurons by interfering with availability of reduced NADH. Chloroquine phosphate has been found to inhibit intracellular oxidation of NADH by melanin.

Chloroquine phosphate binds to neuromelanin and does not inhibit enzymatic synthesis of iron into biologically essential compounds. It not only prevents incorporation of iron into neurons, but also inhibits the release of iron from intracellular iron pools. In addition chloroquine phosphate has been found to heighten an astrocytic immune response against accumulation of extracellular protein deposits in the brain contributing to Alzheimer's Disease.

The (−) isomer of chloroquine is an even more effective neuromelanin binder than racemic chloroquine because it breaks down less peripherally, has a longer half-life and lower clearance, and so is more available to cross the blood brain barrier, as well as having a stabilizing effect on DNA. It is therefore preferred for use in this invention.

### EXAMPLES

### Example 1

Enantiomers of chloroquine phosphate were isolated according to the procedure of Stalcup, A. M. et al. (1996), Analytical Chemistry 68:2248-50. Comparisons of these enantiomers with respect to ability to inhibit diamine oxidase and bind to neuromelanin are performed in vitro. Results show significantly enhanced ability of the active enantiomer in both assays to inhibit diamine oxidase and bind neuromelanin.

### Example 2

A within-subjects, open labeled, parallel study is performed to evaluate the efficacy of CQ and enantiomeric CQ (test compounds) for the treatment of motor disorders in adults having a diagnosis of Idiopathic Parkinson's Disease (IPD) and Symptomatic Parkinson's Disorders, using the Unified Parkinson's Disease Rating Scale and the Tinetti Gait and Balance Assessment Tool for assessment pre-treatment, during treatment, and two weeks post-treatment. The treatment period assesses the safety and durability of response for up to eight weeks. An initial two-week pre-treatment period establishes each participant's baseline neurophysiological and well-being measures. A final evaluation, administered following a two-week treatment withdrawal period, evaluates each participant for symptom restoration.

Thirty adults 30-75 years of age, including fifteen subjects having a confirmed diagnosis of stage I-III IPD, designated Group I, and 15 subjects having a diagnosis of Symptomatic Parkinson's Disorders resultant from vascular disorders, multi-infarct state, hypoxia, normal pressure hydrocephalus and/or postencephalitis, designated Group II, receive a reverse titration of the test medication during the first week of treatment. This is followed by a one-time-per-day maintenance dose of 155 mg per day taken with the evening meal.

During the initial 24-hour treatment period, subjects are instructed to take 155 mg of the test medication four times per day. On study days 2 and 3, subjects are instructed to take 155 mg of test medication 3 times per day. On study days 4, 5 and 6, subjects are instructed to take 155 mg of test medication 2 times per day. On study day 7, subjects are instructed to take 155 mg of the test medication daily with their final meal of the day. On treatment day 10, physicians determine final maintenance dose to be taken each evening with the subject's final meal of the day for the duration of the treatment period. The maintenance dose may be kept at 155 mg test medication per day or adjusted to a lower or higher dose, e.g. down to 100 mg if the subject is showing improvement but having gastrointestinal or other discomforts. The dose is increased up to 200 mg or 255 mg per day if the subject has not experienced symptom relief.

Improvements in pre-treatment (baseline) scores on the above-described or similar measuring instruments, and/or decline in function and score values following the medication withdrawal period are used for assessment. Subjects are checked for the occurrence of adverse events during the study three times per week. Laboratory evaluations (chemistry and hematology profiles) are performed at pre-treatment screening and during the treatment period on days 10, 28 and 56, and also during the two-week post-treatment exit evaluation.

Pre-treatment (baseline) measurements are taken on three separate occasions, at three different times of the day, morning, noon and evening, during the initial two-week pre-treatment evaluation period. The pre-treatment scores are averaged to determine each patient's baseline neurophysiological and well-being measurements. Five separate neurophysiological and well-being evaluations are administered on treatment days 7, 14, 28, 42 and 56. Medication is discontinued immediately after the neurophysiological and well-being evaluation administered on treatment day 56. Patients are seen for one additional exit interview including complete neurophysiological, well-being, and laboratory evaluations two weeks after the experimental treatment is discontinued.

Baseline (averaged) scores obtained during the two-week pre-treatment period are compared to scores obtained during treatment days 7, 14, 28, 42 and 56 to determine any changes in patient status throughout the treatment period. The final two-week post-treatment evaluation scores are used to determine a lingering effect by comparing the two-week post-treatment evaluation scores to the pre-treatment baseline measurements. Improvement of motor symptoms from pre-treatment, during treatment and post-treatment are evaluated, as well as improved well-being. Within-group improvement scores are analyzed using a t-test of differences for scores from the pre-test condition to the post-test condition using a p value of 0.05. Variables are summarized by treatment group according to subgroups of gender, race, and age. Treatment groups are further compared using the Cochran-Mantel-Haenszel test with stratifications by the above variables.

Changes from pre-treatment evaluations to days 7, 14, 28, 42 and 56 in each clinical sign and symptom are summarized by treatment group. The two treatment groups are compared with respect to the percentage of subjects who showed either resolution or improvement in the signs and symptoms among the subjects presenting with the signs and symptoms using Fisher's exact test. Potentially clinically significant laboratory values and mean changes from baseline of vital signs data are summarized within both treatment groups. Times to resolution/improvement of symptoms after treatment are also summarized by treatment group and compared using the log rank test. Subject satisfaction data and subject symptoms collected from questionnaires are also summarized by treatment group and analyzed. Based on an adverse event rate of 3%, the treatment group sizes used provide approximately 80% power to detect significance difference at the 0.05 (two-tailed) significance level.

Significant improvement in symptoms and halting of progression of symptoms both during and post-treatment is observed.

Preliminary clinical studies yielded the following results:

THE USE OF CHLOROQUINE DIPHOSPHATE FOR
MULTIPLE SYMPTOM ATROPHY (MSA)
Protocol # PD/CQ CASE #1 (MSA)

| | | | | Symptoms | Diagnosed | Present | | |
|---|---|---|---|---|---|---|---|---|
| | | Birth | | First | by | Stage | Height | Weight |
| ID # | Initials | Date | Sex | Noticed | Physician | PD | (inches) | (pounds) |
| 70101 | DKS | Jan. 26, 1935 | M | n/a | May 1998 | V | 71″ | 230 |

| | BASELINE | Rx DAY 14 | BL- Day |
|---|---|---|---|
| UPDRS | SCORES | SCORES | 14 = change |
| ADL OFF | 34 | 32 | 2 |
| ADL ON | 34 | 32 | 2 |
| MOTOR OFF | 50 | 46 | 4 |
| MOTOR ON | 47 | 44 | 3 |
| ADL + MOTOR | 84 | 78 | 6 |
| OFF | | | |
| ADL + MOTOR | 81 | 76 | 5 |
| ON | | | |
| TOTAL UPDRS | 85 | 82 | 3 |

| TIMED TAPPING | BASELINE | Rx DAY 14 | Day 14-BL |
|---|---|---|---|
| Right Hand “OFF” | 44 | 54 | +10 |
| Right Hand “ON” | 47 | 51 | +4 |
| Left Hand “OFF” | 40 | 48 | +8 |
| Left Hand “ON” | 46 | 49 | +3 |

Case #1 was the first patient to be administered CQ+brain targeting agent (BTA; cimetidine) having a confirmed diagnosis of Multiple Symptom Atrophy (MSA). Motor improvements can be seen in both the timed tapping and UPDRS scales scores between baseline and treatment day 14. On the medication day 35 visit, patient reported less freezing (i.e.—OFF time) during the previous 10 days and an increase in concentration. The patient's speech therapist and physical therapist, both seen bi-weekly, reported respective improvements in speech and range of motion. However, following the day 35 visit, a violation of the protocol occurred that necessitated the disqualification of this patient from enrollment.

THE USE OF CHLOROQUINE DIPHOSPHATE FOR PARKINSON'S PLUS DISORDERS
Protocol # IPDRac519 CASE #2 (Parkinson's w/concurrent dementia)

| | | | | | Diagnosed | Present | | |
|---|---|---|---|---|---|---|---|---|
| | | Birth | | Symptoms | by | Stage of | Height | Weight |
| ID # | Initials | Date | Sex | First Noticed | Physician | PD | (inches) | (pounds) |
| 80103 | RAW | Oct. 27, 1922 | M | 1990 | 1996 | III | 71.5″ | 132 |

| | | | | Rx DAY | |
|---|---|---|---|---|---|
| | BASELINE | Rx DAY 14 | BL- DAY | 56 | BL- Day 56 = |
| UPDRS | SCORES | SCORES | 14 = change | SCORES | change |
| ADL OFF | 14 | 18 | −−4 | 12 | 2 |
| ADL ON | 12 | 15 | −−3 | 11 | 1 |
| MOTOR OFF | 28.5 | 41 | −−12.5 | 30 | 1.5 |
| MOTOR ON | 22.5 | 27.5 | −−5 | 24 | 1.5 |
| ADL + MOTOR OFF | 42.5 | 59 | −−16.5 | 42 | 0.5 |
| ADL + MOTOR ON | 34.5 | 42.5 | −−8 | 35 | −−0.5 |
| TOTAL UPDRS | 39.5 | 52.5 | −−13 | 39 | 0.5 |

| TIMED TAPPING | BASELINE | Rx DAY 14 | Day14-BL | Day 56 | Day 56-BL |
|---|---|---|---|---|---|
| Right Hand “OFF” | 47 | 63 | +16 | 58 | +11 |
| Right Hand “ON” | 59 | 56 | −3 | 64 | +5 |
| Left Hand “OFF” | 52 | 57 | +5 | 69 | +17 |
| Left Hand “ON” | 59 | 53 | −6 | 62 | +3 |

Case #2 enrolled having a confirmed diagnosis of stage III Parkinson's Disease with progressive diminishment in cognitive function (Mini-Mental State Exam score of 24, dementia≦24). Similar to Case #1, this patient appeared to have dramatic improvements in cognition and memory while taking CQ+BTA. Two weeks post withdrawal from CQ, both the patient and his wife reported reemerging difficulty in “word finding” and a significant decline in both concentration and memory. The patient requested to be put back on and resumed taking CQ+BTA a week after withdrawal.

Two properties make CQ a likely agent to improve memory and cognitive function. One being that CQ (especially the (+)-CQ enantiomer) is an acetylcholine esterase inhibitor (AChE), which would augment ACh levels (the memory neurotransmitter) in the brain. While this may have contributed to the patient's increase in memory and cognition, ACh is also known to contribute to Parkinson's disease symptomology. As was observed in the timed tapping (tt) and UPDRS evaluations, there appears to be a significant “motor functional” improvement. The second reason for cognitive improvement could be a result of heightened brain tissue oxygenation while the patient was taking CQ. CQ and hydroxychloroquine (HCQ) are both agents known to increase red blood cell (RBC) 02 absorption and delivery, via mechanisms of a Bohr shift promoted by alterations in physiological pH.

THE USE OF CHLOROQUINE DIPHOSPHATE
FOR ATYPICAL PARKINSON'S DISEASE (APD)
Protocol # IPDRac519 CASE #3 (Atypical Parkinsonian Disorder)

| | | | | Symptoms | Diagnosed | Present | | |
|---|---|---|---|---|---|---|---|---|
| | | Birth | | First | by | Stage | Height | Weight |
| ID # | Initials | Date | Sex | Noticed | Physician | PD | (inches) | (pounds) |
| 80107 | RAC | July 6, 1926 | M | 2001 | April 2001 | II | 69.1″ | 207 |

| | | | | Rx DAY | |
|---|---|---|---|---|---|
| | BASELINE | Rx DAY 14 | BL- DAY | 56 | BL- Day 56 = |
| UPDRS | SCORES | SCORES | 14 = change | SCORES | change |
| ADL OFF | 12 | 9 | 3 | 9 | 3 |
| ADL ON | 12 | 9 | 3 | 9 | 3 |
| MOTOR OFF | 19 | 17.5 | 1.5 | 13.5 | 5.5 |
| MOTOR ON | 20 | 16.5 | 3.5 | 12 | 8 |
| ADL + MOTOR | 31 | 26.5 | 4.5 | 22.5 | 9.5 |
| OFF | | | | | |
| ADL + MOTOR | 32 | 25.5 | 6.5 | 21 | 11 |
| ON | | | | | |
| TOTAL UPDRS | 34 | 27.5 | 6.5 | 24 | 10 |

| TIMED TAPPING | BASELINE | Rx DAY 14 | Day14-BL | Day 56 | BL-Day 56 |
|---|---|---|---|---|---|
| Right Hand “OFF” | 84 | 80 | −−4 | 89 | +5 |
| Right Hand “ON” | 61 | 80 | +19 | 68 | +7 |
| Left Hand “OFF” | 92 | 77 | −−15 | 79 | −−13 |
| Left Hand “ON” | 80 | 75 | −−5 | 74 | −−6 |

Case #3 was diagnosed with Parkinsonism less than 1 year. Review of the outside patient records suggests that this patient had an atypical Parkinson's disorder. In addition, there was no significant levodopa response prior to acceptance into the study. In fact, this patient subsequently failed to follow instructions regarding co-administration of Sinemet with CQ+BTA and stopped all other anti-Parkinson medications for about 10 days without any deterioration in Parkinsonian symptoms. This is a significant indication that this patient does not have the idiopathic form of Parkinson's disease.

Case #3 was administered treatment day 14 UPDRS functional off/on evaluations on 11-21-01. Patient was doing well even while he had erroneously discontinued taking Sinemet two weeks prior when he began taking CQ+BTA. Patient was instructed to resume taking Sinemet CR 50/100 three times per day and Sinemet 10/100 twice daily. No adverse events or motor improvements were noted during the treatment day 35 visit, but patient reported a sight increase in mental activity. Patient was given an increased dose of CQ from 150 mg to 200 mg per day, following his day 35 visit. By treatment day 56 patient reported that since increasing his dose of CQ, he has “come alive.” Patient requested to be put back on and resumed taking CQ 200 mg/day+BTA 400 mg/day immediately following his exit evaluation visit.

In the case of Multiple Symptom Atrophy (Case #1), Parkinson's Plus Syndromes (Case #2) and/or other non L-Dopa responsive Atypical Parkinsonian Disorders (Case #3) the compositions comprised of Chloroquine diphosphate with BTAs can be used to effectively improve cognition, alleviate motor symptoms and attenuate the progression of these disorders when administered in dosages similar to dosages that are required to treat Idiopathic Parkinson's Disease.

Our study results demonstrate synergistic interactions between CQ and various other Parkinson's medications. Patients enrolled in the study protocol reported experiencing an attenuation in motor fluctuations, a diminishment of “freezing” and a significant reduction in “OFF” time, while they were taking CQ+BTA. The remarkable consideration was that these improvements were reported to have emerged and were sustained following some very dramatic reductions made in concomitant Parkinson's disease medications.

While the invention has been described in specific terms, it is not to be limited to the description herein but is to be afforded the full scope of the appended claims and all equivalents thereto. For example, other neuromelanin-binding compounds and complexes containing the quinoline ring structure known to the art are equivalent to those specifically described, as are other modifications to the compositions to enhance bioavailability, crossing the blood/brain barrier, biological half-life, or other desirable properties.

1. A composition useful for increasing cellular respiration of melanized catecholamine neurons comprising an active ingredient which is a compound selected from the group consisting of: 7-chloro-4-(4-diethylamino-1-methylbutylamino)quinoline (chloroquine); 7-fluoro-4-(4-diethylamino-1-methylbutylamino)quinoline; 4-(4-diethylamino-1-methylbutylamino)quinoline; 7-hydroxy-4-(4-diethylamino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethylamino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethylamino-1-butylamino)quinoline; 4-(1-carboxy-4-diethylamino-1-butylamino)quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethylamino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-chloro-4-(4-ethyl-(2-hydroxyethyl)-amino-1-methylbutylamino)quinoline (hydroxychloroquine); 7-fluoro-4-(4-ethyl-(2-hydroxyethyl)-amino-1-methylbutylamino)quinoline; 4-(4-ethyl-(2-hydroxyethyl)-amino-1-methylbutylamino)quinoline7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino)quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino)-6-methoxydihydrochloride quinoline; 1-acetyl-1,2,3,4-tetrahydroquinoline; 8-[4-aminopentyl)amino]-6-methoxyquinoline dihydrochloride; 1-butyryl-1,2,3,4-tetrahydroquinoline; 3-chloro-4-(4-hydroxy-α,α′-bis(2-methyl-1-pyrrolidinyl)-2,5-xylidinoquinoline, 4-[(4-diethylamino)-1-methylbutyl)amino]-6-methoxyquinoline; 3-fluoro-4-(4-hydroxy-α,α′-bis(2-methyl-1-pyrrolidinyl)-2,5-xylidinoquinoline, 4-[(4-diethylamino)-1-methylbutyl)amino]-6-methoxyquinoline; 4-(4-hydroxy-α,α′-bis(2-methyl-1-pyrrolidinyl)-2,5-xylidinoquinoline, 4-[(4-diethylamino)-1-methylbutyl)amino]-6-methoxyquinoline; 3,4-dihydro-1-(2H)-quinolinecarboxyaldehyde; 1,1′-pentamethylenediquinoleinium diiodide; and 8-quinolinol sulfate, racemic mixtures, and enantiomers thereof, phosphate salts and other suitable pharmaceutical salts thereof, and mixtures thereof, said compounds being covalently linked or complexed or mixed with an adjuvant.

2. The composition of claim 1 wherein said active ingredient is selected from the group consisting of: chloroquine, chloroquine phosphate, hydroxychloroquine, and racemic mixtures and enantiomers thereof, covalently linked, mixed, or complexed with an adjuvant, acceptable pharmaceutical salts thereof, and mixtures of the foregoing, said active ingredient and adjuvant being present in amounts effective to increase melanized catecholamine neurons.

3. The composition of claim 1 wherein said active ingredient is present in an amount between about 100 and about 500 mg.

4. The composition of claim 1 wherein said active ingredient is present in an amount between about 100 and 200 mg.

5. The composition of claim 1 wherein said adjuvant is a peripheral membrane protective agent.

6. The composition of claim 5 wherein said adjuvant is a retinal protective agent.

7. The composition of claim 5 wherein said composition is provided in the form of a time-release preparation, and said peripheral protective agent is not complexed or covalently bound to said active ingredient.

8. The composition of claim 5 wherein said peripheral protective agent is selected from the group consisting of calcium citrate, calcium gluconate, calcium lactate, and calcium phosphate.

9. The composition of claim 8 wherein said peripheral protective agent also comprises vitamin D.

10. The composition of claim 8 wherein calcium ion is present in an amount between about 1000 mg to about 2000 mg.

11. The composition of claim 9 wherein Vitamin D is present in an amount between about 700 and about 900 IU.

12. The composition of claim 8 designed to release said peripheral protective agent from about 1.5 to about 3 hours prior to release of said active ingredient.

13. The composition of claim 1 wherein said adjuvant is a peripheral metabolism inhibitor that inhibits peripheral metabolism of said active ingredient.

14. The composition of claim 13 wherein said metabolism inhibitor is an inhibitor of cytochrome P450 2D6 and/or 3A enzyme.

15. The composition of claim 13 wherein said metabolism inhibitor is a cytochrome (CYP) 2D6 enzyme inhibitor selected from the group consisting of amiodarone, celecoxib, chlorpheniramine, cimetidine, clomipramine, fluoxetine, levomepromazine, metoclopramide, mibefradil, moclobemide, paroxetine, quinidine, ranitidine, ritonavir, sertraline, terbinafine, racemic mixtures and enantiomers and acceptable pharmaceutical salts of the foregoing.

16. The composition of claim 13 wherein said metabolism inhibitor is a cytochrome P450 3A enzyme inhibitor selected from the group consisting of delavirdine, indinavir, nelfinavir, saquinavir, amiodarone, cimetidine, ciprofloxacin, clarithromycin, diethyl-dithiocarbamate, diltiazem, erythromycin, fluconazole, fluvoxamine, itraconazole, ketoconazole, mifepristone, nefazodone, mifepristone, norfloxacinm, norfluoxetine, racemic mixtures and enantiomers, and acceptable pharmaceutical salts of the foregoing.

17. The composition of claim 13 wherein said metabolism inhibitor is selected from the group consisting of amiodarone, cimetidine, racemic mixtures and enantiomers, and acceptable pharmaceutical salts of the foregoing.

18. The composition of claim 1 also comprising a peripheral protective agent.

19. The composition of claim 18 in the form of a time-release preparation.

20. The composition of claim 19 designed to release said metabolism inhibitor about 1.5 to about 2 hours after said peripheral protective agent and about 1 hour before said active ingredient.

21. The composition of claim 1 wherein said adjuvant is an enhancing agent.

22. The composition of claim 21 wherein said enhancing agent is a histamine H₁ receptor antagonist.

23. The composition of claim 21 wherein said enhancing agent is a first-generation histamine H₁ receptor antagonist.

24. The composition of claim 23 wherein said first-generation histamine H₁ receptor antagonist is selected from the group consisting of carbinoxamine maleate, clemastine, diphenhydramine, dimenhydrinate, pyrilamine maleate, tripelernamine, chlorpheniramine maleate, brompheniramine maleate, hydroxyzine hydrochloride, hydroxyzine pamoate, cyclizine hydrochloride, cyclizine lactate, meclizine hydrochloride, promethazine hydrochloride, and racemic mixtures and enantiomers, and acceptable pharmaceutical salts of the foregoing therapeutic moieties.

25. The composition of claim 24 in the form of a time-release preparation.

26. The composition of claim 25 designed to release said first-generation histamine H₁ receptor about concurrently with said active ingredient.

27. The composition of claim 21 wherein said enhancing agent is a second-generation histamine H₁ receptor antagonist.

28. The composition of claim 27 wherein said second generation histamine H₁ receptor antagonist is selected from the group consisting of acrivastine, cetirizine hydrochloride, astemizole, loratadine, terfenadine, racemic mixtures and enantiomers, and suitable pharmaceutical salts of the foregoing therapeutic moieties.

29. The composition of claim 28 in the form of a time-release preparation.

30. The composition of claim 29 designed to release said second-generation histamine H₁ receptor about concurrently with said active ingredient.

31. The composition of claim 1 wherein said active ingredient is covalently linked to a lactotransferrin antibody.

32. The composition of claim 1 wherein said active ingredient is a (−)-enantiomer thereof.

33. The composition of claim 32 wherein said active ingredient consists essentially of an effective amount of a (−)-enantiomer thereof, and a lesser amount of a (+)-enantiomer thereof.

34. The composition of claim 33 wherein said (+)-enantiomer is from about 0% to about 20% of the total (+)-enantiomer and (−)-enantiomer.

35. The composition of claim 1 wherein said active ingredient is chloroquine.

36. The composition of claim 1 wherein said active ingredient is chloroquine phosphate.

37. The composition of claim 1 wherein said active ingredient is hydroxychloroquine.

38. The composition of claim 1 wherein said active ingredient is covalently linked with a lipophilic moiety.

39. The composition of claim 1 wherein said adjuvant is selected from the group consisting of neural protective compounds other than said active ingredient, dopamine and dopamine agonists, and free radical deactivators.

40. The composition of claim 1 wherein said adjuvant is an antioxidant selected from the group consisting of probucol, pycnogenol, Vitamin C, Vitamin E, butylated hydroxytoluene (BHT), butylated hydroxyanisole (BHA), melatonin, and superoxide dismutase.

41. A method of making a pharmaceutical composition of claim 1 effective for increasing cellular respiration of melanized catecholamine neurons, said method comprising:
(a) providing a neuromelanin-binding agent as an active ingredient;
(b) providing an adjuvant for said active ingredient;
(c) providing a suitable pharmaceutical carrier; and
(d) mixing, complexing or covalently bonding said active ingredient with said adjuvant, and compounding said active ingredient and adjuvant with said pharmaceutical carrier.

42. The method of claim 41 wherein said active ingredient is selected from compounds of the group consisting of chloroquine, chloroquine phosphate, hydroxychloroquine, enantiomers, racemic mixtures, and suitable pharmaceutical salts thereof.

43. The method of claim 41 wherein said adjuvant is selected from the group consisting of brain-targeting agents, peripheral membrane protectors, peripheral metabolism inhibitors, enhancing agents, racemic mixtures, enantiomers, and acceptable pharmaceutical salts thereof, and mixtures of the foregoing;

44. A method for treating a condition selected from the group consisting of idiopathic Parkinson's disease, multiple symptom atrophy associated with Parkinson's disease, Parkinson's Plus Syndrome, Atypical Parkinsonian Disorders, on-off syndrome associated with treatment with dopamine or a dopamine agonist, conditions characterized by nigrostriatal degeneration, and vascular Parkinson's disease, said method comprising administering to said patient, in an effective regimen and amount, a composition of claim 1.

45. A method for reducing the amount of dopamine or dopamine agonists used to treat a patient suffering from a condition selected from the group consisting of idiopathic Parkinson's disease, multiple symptom atrophy associated with Parkinson's disease, Parkinson's Plus Syndrome, Atypical Parkinsonian Disorders, on-off syndrome associated with treatment with dopamine or a dopamine agonist, conditions characterized by nigrostriatal degeneration, and vascular Parkinson's Disease, said methods comprising administering to said patient, in an effective regimen and amount, a composition of claim 1.
